(19) United States
(12) Patent Application Publication
(10) Pub. No.: US 2021/0346600 A1
(43) Pub. Date: Nov. 11, 2021

(54) Insertion Device

(71) Applicant: EMED Technologies Corporation, El Dorado Hills, CA (US)

(72) Inventors: Paul Lambert, El Dorado Hills, CA (US); Carlos Gutierrez, El Dorado Hills, CA (US)

(21) Appl. No.: 17/285,876

(22) PCT Filed: Oct. 16, 2019
(86) PCT No.: PCT/US2019/056570

§ 371 Date: Apr. 15, 2021

Related U.S. Application Data
(60) Provisional application No. 62/746,351, filed on Oct. 16, 2018.

(51) Int. Cl. A61M 5/158

(52) U.S. Cl. CPC A61M 5/158; A61M 2005/1585

(57) ABSTRACT

An insertion device for transcutaneous placement of a medical needle through the skin of a patient includes a cylindrical housing with a shell with a distal opening and a proximal opening. A carriage is slidably received within the shell between a distal position and a retracted position. The carriage includes elements to secure a needle assembly.

### CROSS-REFERENCE TO RELATED APPLICATIONS

The present application claims the benefit of and priority to U.S. Provisional Patent Application No. 62/746,351 entitled “Insertion Device” filed on Oct. 16, 2018 2, 2008, the foregoing application being incorporated herein, by reference, in its entirety.

### FIELD

The disclosure relates generally to medical infusion systems. The disclosure relates specifically to insertion of a needle at a selected site within the body of a subject for subcutaneous, intravenous, intramuscular, or intradermal delivery of a drug to the subject.

### BACKGROUND

Medical needles are widely used in the course of patient treatment, particularly for delivery of selected medications. In one common form, hollow hypodermic needles are employed for transcutaneous delivery of a selected medication from a syringe or the like. In another common form, insertion needles are employed for transcutaneous placement of a soft and relatively flexible tubular cannula, followed by insertion needle removal and subsequent infusion of medical fluid to the patient through the cannula. Referring to FIG. 1, a needle assembly 600 contains a main body 601, a pair of stabilization wings 603 attached to the main body 601, a medication supply tubing 602 is embedded into the main body 601 and communicates with a needle 605. Of course, a needle assembly 600 may be of simple or basic sort that includes only a needle 605 and, optionally, a main body 601 (whether or not the main body is aligned longitudinally with the needle 605) and/or medication supply tubing 602.

Certain therapies such as immunoglobulin therapy can be self-administered by a patient in the comfort of the patient's home. Infusion therapies require the user to insert a needle into the patient's body. While some patients have no difficulty self-inserting needles or receiving needles in their body, other patients are sensitive to the pain of the injection or are uncomfortable seeing needles or injecting needles into their body. In particular, many children have difficulty receiving infusion therapy due to the pain of needle insertion or fear of needles.

The method in which the needle is injected into the tissue is relevant to preventing pain. Research has shown that the speed of the insertion of the needle is important to protect the tissue layers from rupturing and reduce the pain of the insertion. When a needle is slowly inserted into the tissue, more rupture events are observed in the tissue as compared to the rapid insertion of a needle into tissue.

U.S. Pat. No. 6,830,562 describes an injection device to facilitate delivery of a dose of medicament to a patient through a hypodermic needle. The injection device can be administered by the patient themselves and can provide both psychological and physical advantages to patients. The device comprises a housing, a coil spring, and a safety device to offer a solution for automatic injection of medication. However, the apparatus is relatively complicated to manufacture and further the device has to be loaded manually by the patient by a rather complicated procedure.

The present invention relates to an automatic insertion device, particularly for use with a subcutaneous infusion set, for quickly and easily placing an insertion needle and related cannula through the skin of a patient at the correct insertion angle, and with a speed and force of insertion which minimizes patient discomfort.

### SUMMARY

An object of the invention is to provide an improved insertion device which is easy to be manufactured and which is easily used by a patient.

In accordance with the invention, an insertion device is provided for quick and easy transcutaneous placement of a medical needle through the skin of a patient, particularly such as an insertion needle and related cannula of a subcutaneous infusion set. The insertion device is designed to place the needle through the skin at a selected place and with a controlled force and speed of insertion to ensure proper needle placement with minimal patient discomfort.

In one aspect, an insertion device comprises a cylindrical housing with a shell with a distal opening and a proximal opening; a carriage slidably received within the shell between a distal position and a retracted position; wherein said carriage has elements to secure a needle assembly.

The insertion device may include a drive for biasing the cylindrical housing relative to the carriage. In one embodiment, the drive may comprise a spring.

The insertion device may also include a trigger-type actuator to control the release of the carriage. The trigger-type actuator may include a button and a trigger spring.

Optionally, the carriage includes a pillar in the center. A pair of fingers may be attached on opposite sides of the pillar at a forward end thereof for releasably receiving and retaining the needle assembly. The carriage may also include a pair of trigger arms that project generally rearwardly from a platform that is adjacent to a rear end of the pillar. The trigger arms may include out-turned trigger fingers—i.e., extending laterally away from a centerline of the trigger arm—at a rear or distal end of the trigger arm. The carriage may also include at least one barb and, in some instances, a pair of barbs that project generally forwardly from the platform and include edges at the front or proximal end of the platform to limit the slide distance of the carriage and prevent it from disengaging from the housing. The carriage also may comprise a frame at the front end of the pillar to accommodate a main body of the needle assembly.

Optionally, the housing includes one or more guide slots on an inner wall configured to guide the carriage. A bottom end of the housing may be larger in diameter or width than a diameter or width of a top end of the housing.

The trigger-type actuator may include a button and a trigger spring. The button optional comprises a cylindrical sleeve with a tapered or ramped leading-edge face configured to engage a ramped outer face or faces of the trigger fingers.

A lower end of the housing may include a curvature to adapt to the skin, the hands, or the fingers of a user or patient. Two inward curving outlines may be formed at the lower end of the housing. Further, the insertion device may also include a curvature to rest a user or patient's thumb and middle finger and configured to press against a patient's skin when setting the inserter on the patient's skin. The inserter may include haptic or sensitization points to distract the brain from the needle puncturing the patient's skin.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 shows a needle assembly which can be used in the present invention;

FIG. 2 is an exploded view of an insertion device, according to one embodiment of the present invention;

FIG. 3 is a perspective view of an insertion device;

FIG. 4 is a cross-sectional view of an insertion device with a carriage in the distal position;

FIG. 5 is a cross-sectional view of an insertion device with a carriage in the proximal position;

FIG. 6 is a bottom view of an insertion device in FIG. 3.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein to describe the insertion device, or any of the relative positions of the components of the present invention, the terms “axial” or “axially” refer generally to a longitudinal axis around which the components of the insertion device are preferably positioned, although not necessarily symmetrically there-around. The term “radial” refers generally to a direction normal to the longitudinal axis. The terms “proximal,” “rearward,” or “backward” refer generally to an axial direction in the direction away from the needle. The teams “distal,” “front,” “frontward,” “ ” or “forward” refer generally to an axial direction in the direction close to the needle.

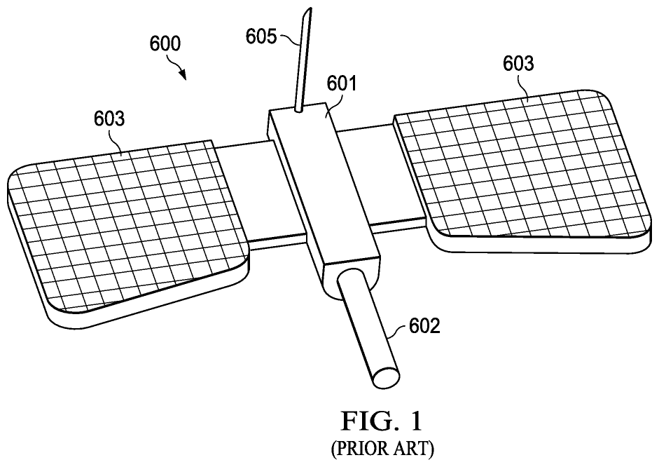

As shown in the exemplary drawings, an insertion device is provided for quick and easy transcutaneous placement of a medical needle, particularly such as an insertion needle assembly 600 as depicted in FIG. 1, although the insertion device may be used with any type of needle. The insertion device includes a trigger-type actuator mechanism for transcutaneous placement of the needle assembly 600 with a controlled speed and force.

The insertion device of the present invention represents a simple device which can be used by the patient to quickly and easily place the needle assembly 600 at a selected medication insertion site. The insertion device is designed to project the infusion set toward the patient's skin at a controlled force and speed for quickly piercing the skin in a manner ensuring proper placement of the needle assembly 600, while minimizing patient anxiety and/or discomfort.

Improper and/or partial placement of the insertion needle is thus avoided.

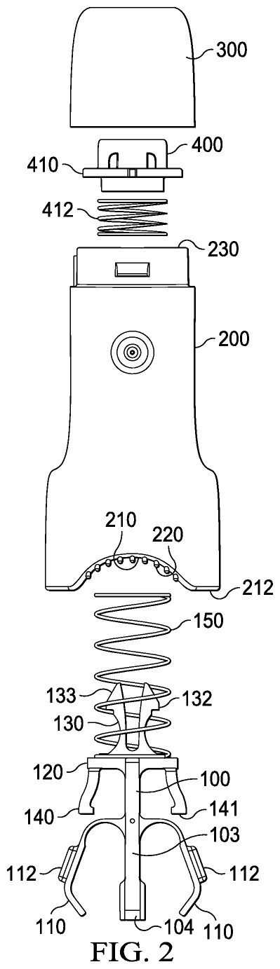

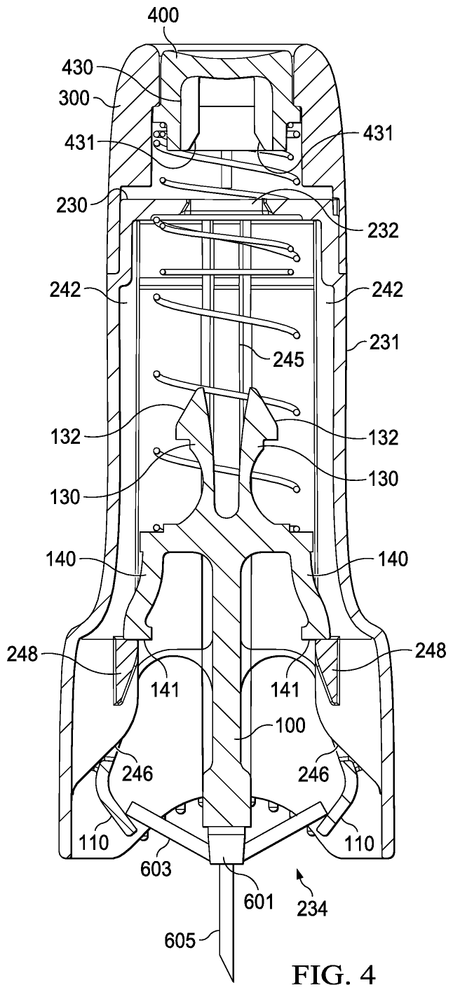

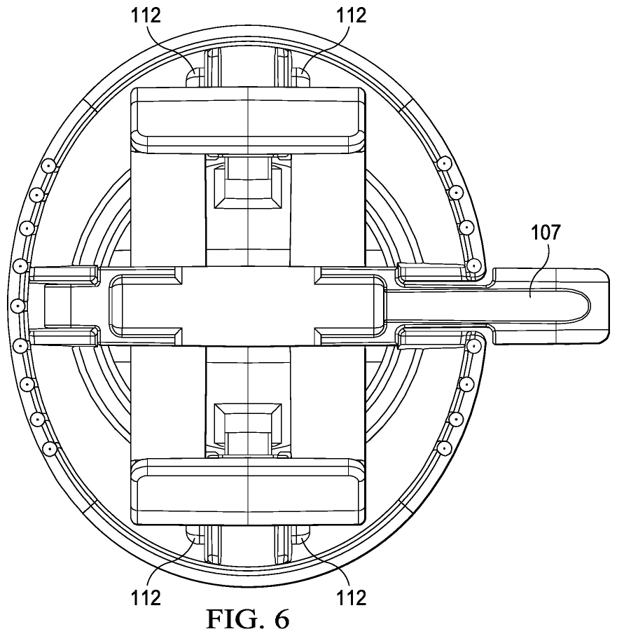

As shown in an embodiment in FIGS. 2-6, the insertion device comprises a housing 200 (FIG. 2), which optionally may be cylindrical or any other shape. The housing 200 comprises a shell 231 with a distal opening 232 and a proximal opening 234 (FIG. 4); the housing 200 may be made of any of a number of materials including plastics, metals, and glass. In an embodiment, the housing 200 is made of plastic.

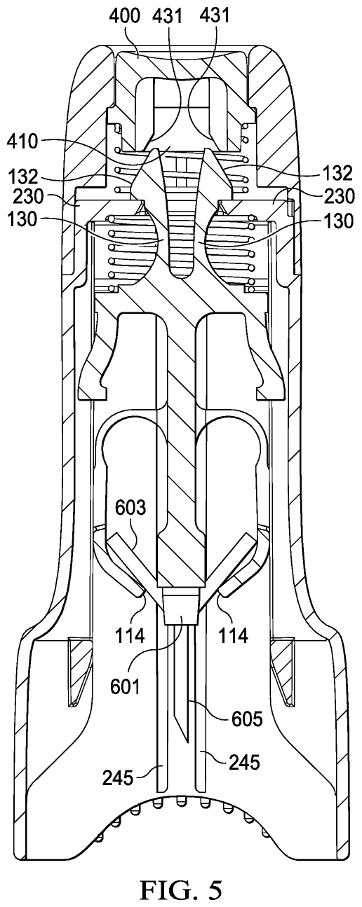

An interior space defined by the shell 231 can receive a carriage 100 such that the carriage 100 can longitudinally slide within the shell 231 between a distal position (FIG. 4) and a proximal position (FIG. 5).

The carriage 100 may include a pillar 103 in or near a center of the carriage 100. The carriage also may include at least one and, in some examples, a pair of fingers or a plurality of fingers 110 attached to and extending laterally away from the pillar 103. The fingers 110 may be positioned on opposite sides of the pillar 103, substantially equidistantly around the pillar (within +/−10 degrees of equidistant), or radially around the pillar 103, such as at 180 degrees, 120 degrees, 90 degrees, 60 degrees and so forth depending on the number of fingers 103 that are present. The fingers 103 may be positioned at a forward or proximal end of the carriage 100 and configured to releasably receive and retain the insertion needle assembly 600. The fingers 110 may be made of a flexible material (including plastic and thin metal) such that they can expand and draw back radially away from and towards the pillar 103 in a manner to be described in more detail herein.

The carriage 100 may also include at least one and, in some examples, two or more trigger fingers 132 at a rear or distal end of the carriage 100 spaced apart from the front or the proximal end of the carriage 100. The trigger fingers 132 may be configured to cooperate with the tapered or ramped leading-edge face of the trigger actuator or button 400 (FIGS. 4 and 5) that may be mounted on a rear or distal end of the housing 200. The trigger fingers 132 interact with the shoulder 230 of the distal opening 232 to hold the carriage 100 in a retracted position (FIG. 5) in which the needle 605 is sheathed within the housing 200 against the force of a compressed drive spring 150. A trigger button 400 of the actuator assembly is configured to or adapted for fingertip depression to compress the trigger fingers 132 so as to release them from the shoulder 230 of the distal opening 232 and thereby release the carriage 100 for spring-loaded travel toward the forward or proximal position and corresponding transcutaneous placement of the insertion needle assembly 600 through the patient's skin.

The carriage 100 additionally may include at least one and, in some examples, a pair or a plurality (which encompasses a pair or more) of trigger arms 130 which project generally rearwardly from a platform 120 that is adjacent to a rear or distal end of the pillar 103. The trigger arms 130 may include trigger fingers 132 that include out-turned, i.e., structures that extend laterally away from a centerline of the carriage 100 proximate a rear or distal end of the trigger fingers 132. The trigger fingers 132 may include a distal end, a ramp portion that extends proximally and laterally outwardly from the distal end, and a step or tooth at a proximal end of the ramp portion. The trigger fingers 132 are adapted and sized for partial radial compression toward each other and the centerline of the carriage 100 as the trigger fingers 132 pass through the distal opening 232 of cylindrical housing 200 when the carriage 100 is displaced from the distal position to the retracted position. As the retracted position is reached, the trigger fingers 132 are spring-loaded by the resiliency of the trigger arms 130 to move outwardly to be hooked by an outer face of a shoulder 230 of the distal opening 232 of the cylindrical housing 200. In this position, as shown in FIG. 5, the triggers fingers 132 retain the carriage 100 in the retracted position.

A drive spring 150 may be mounted within the cylindrical housing 200 to react between the trigger-type actuator assembly 410 and the carriage 100. The drive spring 150 may be a coil spring positioned between the platform 120 and an inner face of the shoulder 230 of the distal opening 232 of the cylindrical housing 200. The drive spring 150 optionally may partially or fully encompass and/or encircle the trigger arms 130. The drive spring 150 (FIG. 2) normally biases the carriage 100 toward the proximal or extended position.

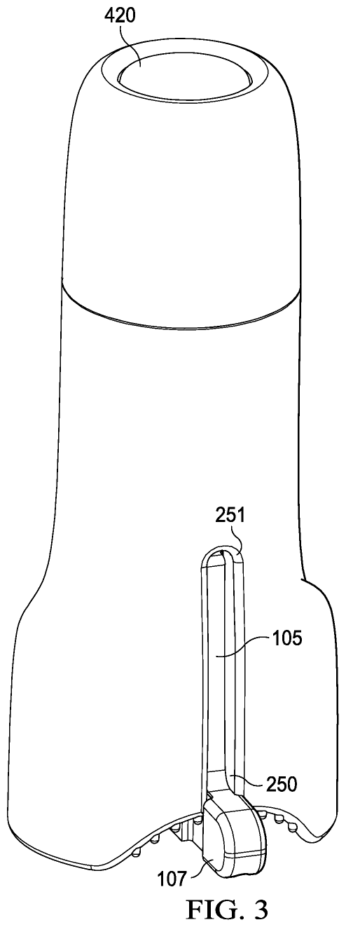

The carriage 100 optionally includes a pushing handle 107 (FIG. 3) attached on the pillar 103 can be pressed rearwardly or distally along the housing 200 to move the carriage 100 from the extended position in FIG. 4 to the retracted position in FIG. 5. In the process of retracting the needle 605 and the carriage 100 with the pushing handle 107, the trigger fingers 132 pass through a distal opening 232 of the cylindrical housing 200 to engaging a shoulder 230 of the distal opening 232. In this regard, the trigger fingers 132 have ramped outboard faces 133 (FIG. 2) to accommodate movement of the trigger fingers 132 radially toward each other as they pass through the distal opening 232. When the trigger fingers 132 pass entirely through the distal opening 232, the spring resilience of the trigger arms 130 is sufficient to spread the trigger fingers 132 so that they engage the shoulder 230. In this retracted carriage position, the drive spring 150 is retained in a compressed condition with the needle assembly 600 withdrawn into the interior of the housing 200 in a spaced relation to the patient's skin.

The trigger-type actuator assembly 410 optionally comprises a button 400, a shoulder (unlabeled, but generally indicated by the lead line for trigger-type actuator assembly 410), and a trigger spring 412 (FIG. 2). The button 400 and the trigger spring 412 may be positioned within or accommodated in a cap 300 at an upper or distal end of the cylindrical housing 200. The shoulder may interact with the cap 300 so as to retain the button 400 within the cap against the urging of the trigger spring 412. The cap 300 and the cylindrical housing 200 may be coupled together through various manners. In various embodiments, the cap 300 and cylindrical housing 200 may be engaged through threads, interference fit, snap fit, or as in FIG. 2 the cap 300 and the cylindrical housing 200 may be engaged by grooves or recesses within an outer surface of the housing 200 and complementary projections in an inners surface of the cap 300 or vice-versa.

The button 400 may comprise a cylindrical sleeve 430 configured to slide within the cap 300 longitudinally. The cylindrical sleeve 430 may include a tapered or ramped leading-edge face 431 for engaging the ramped outboard faces 133 of the trigger fingers 132. The tapered leading-edge face 431 interact with the ramped outboard faces of the trigger fingers 132 to radially compress the trigger arms 130 and release the carriage 100 for spring-loaded travel from the retracted position (FIG. 5) to the extended or distal position (FIG. 4). The button 400 is exposed for fingertip depression at the top of the cap 300 to move the cylindrical sleeve 430 into releasing engagement with the trigger fingers 132.

The trigger spring 412 is mounted within the cap 300 and may comprise a coil spring positioned between an upper face of the shoulder 230 and the button 400 (FIG. 2). The drive spring 150 normally biases the button 400 towards the distal position. However, a fingertip can depress the button 400 at the top of the cap 300 to move the cylindrical sleeve 430 against the trigger fingers 132 and release the carriage 100 from the retracted position (FIG. 5) to the extended position (FIG. 4).

Optionally, the button 400 may be a recessed button with a top surface 420 being lower than a top surface of the cap 300 to prevent accidental release.

The carriage 100 may further include at least one and, in some examples, at least a pair or a plurality (encompassing a pair or more) of barbs 140 that project generally proximately from the platform 120. The barbs 140 may include edges 141 at the front or proximal ends of the barbs 140. These barbs 140 are adapted and sized to fit in the housing 200 with the edges 141 configured to slidably engage slots 242 (FIG. 4) on an inner wall of the housing 200 as the barbs 140 move rearward when the carriage 100 is displaced from the extended position to the retracted position. When the button 400 is depressed to release the carriage 100, the compressed drive spring 150 drives the carriage 100 proximally to the extended position (FIG. 4) and the barbs 140 move with the carriage 100 until the edges 141 are blocked by the bosses 248 on the inner shell of the housing 200. The bosses 248 limit the distance of the carriage 100 and prevent it from extending too far from or completely out of the housing 200. In the reverse as the pushing handle 107 is actuated to retract the carriage 100, the trigger fingers 132 are spring-loaded by the resiliency of the trigger arms 130 to move outwardly to be hooked by outer face of the shoulder 230 of the proximal opening 232 of cylindrical housing 200. In this position, as shown in FIG. 5, the triggers fingers 132 retain the carriage 100 in the retracted position.

In order to limit the sliding of the carriage 100 in the housing 200, the housing 200 may include guide slots 245 on the inner wall of the housing 200. The guide slots 245 may be parallel to the longitudinal axis of the housing 200. Optionally, the pillar 103 may have a rectangular shape, and the two sidewalls 105 (FIG. 3), which optionally may not connect to the fingers 110, fit in two of the guide slots 245 respectively. Each of the fingers 110 may include a convex shape and a through groove 112 configured to engage a guide wedge 246 on the inner wall of the housing 200. The pair of barbs 140 are also limited by a pair of guide slots 242, the bosses 248 are located in the guide slots 242, limiting the potential travel and movement of the pair of barbs 140. In other words, these various structures act to constrain the movement of the carriage 100 and, by extension, the needle 605 in a controlled and predictable manner.

Optionally, a bottom or proximal end of the housing 200 is larger in width or diameter than a width or diameter of a top or distal end of the housing 200. The bottom end of the housing 200 is big enough to accommodate the fingers 110 when the carriage 100 is in the distal or retracted position. The pushing handle 107, as discussed, may be attached on the side of pillar 103, and the pushing handle 107 may extend through a through groove 250 (FIG. 3) in the housing 200. The through groove 250 may be provided along one of the guide slots 245 to allow the pushing handle 107 to press rearwardly against the carriage 100 to move the carriage to the retracted position (FIG. 4). Optionally, the through groove 250 may include a front edge 251 proximate a rear or distal end of the through groove 250 to limit the backward distance of travel of the pushing handle 107 and, in turn, the carriage 100.

In an embodiment, a frame 104 is formed at the front end of the pillar 103 to accommodate the main body 601 of the needle assembly 600. Optionally, the frame 104 is a recess within the pillar 103 configured to receive the main body 601 of the needle assembly 600 therein. Upon using the insertion device, the carriage 100 is in the distal position. The main body 601 of the needle assembly 600 is in the frame 104 and the two wings 603 are arranged at the intervals between the fingers 110 and the pillar 103 respectively (FIG. 4); The pushing handle 107 is moved rearwardly—to move the carriage 100 to the retracted position. The fingers 110 move backward, the fingertips 114 of fingers 110 engage the guide wedge 246 on the inner wall of the housing 200, radially compressing the fingers 110 towards the pillar 103 because of the reduced space of the housing 200. The two wings 603 are gripped between the fingers 110 and the pillar 103 respectively, such that the needle assembly 600 is on the locked position (FIG. 5).

The lower or proximal end of the housing 200 has a curvature shaped to comfortable adapt the housing 200 to a patient's skin. For example, two inward curving outlines 210 may be formed at the lower end of the housing 200. Further, the housing 200 may include a curvature to rest a patient or user's thumb and middle finger to press against skin when setting the inserter on the patient's skin. The housing 200 may including haptic or sensitization points 220 to distract the patient from the actual needle puncture.

After the needle assembly 600 is arrange in the locked or retracted position (FIG. 5), the user can set the inserter on the patient's skin using one hand, with the housing 200 oriented generally perpendicular to the skin. The user then presses the button 400 to release the carriage 100 from the retracted position to the extended position (FIG. 4). The needle assembly 600 rapidly travels with carriage 100 with a controlled speed and force of insertion to ensure penetration of the patient's skin with minimal discomfort. The fingers 110 radially expand by resiliency to release the wings 603 of the needle assembly 600 because of the increased space for the fingers 110 to expand when the wings 603 move frontward. The inserter may then be reused.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. An insertion device for receiving and moving a medical needle from a retracted position in which the medical needle is housed therein to an extended position in which the medical needle is exposed to a skin of a patient, the insertion device comprising:
a housing including a shell with a distal opening and a proximal opening spaced apart from the distal opening;
a carriage configured to be slidably received within the shell between an extended position and a retracted position;
wherein the carriage includes elements to secure a needle assembly.

2. The insertion device of claim 1, further comprising a drive configured to bias the carriage relative to the housing.

3. The insertion device of claim 2, wherein the drive comprises a spring.

4. The insertion device of claim 1, further comprising a trigger-type actuator configured to release the carriage from the retracted position.

5. The insertion device of claim 1, wherein the carriage comprises:
a platform; and,
a pillar coupled to the platform and extending away from the platform towards the proximal opening.

6. The insertion device of claim 5, wherein the pillar comprise a frame configured to receive the medical needle;
at least one finger coupled to the pillar and extending laterally away from the pillar, the at least one finger configured to releasably receive and retain the medical needle.

7. The insertion device of claim 6, wherein the at least one finger comprises a plurality of fingers spaced substantially equidistantly around the pillar.

8. The insertion device of claim 5, wherein the carriage further comprises at least one trigger arm coupled to the platform and extending away from the platform towards the distal opening.

9. The insertion device of claim 8, wherein the at least one trigger arm comprises a plurality of trigger arms.

10. The insertion device of claim 8, wherein the at least one trigger arm includes a trigger finger spaced distally from the platform.

11. The insertion device of claim 10, wherein the at least one trigger finger includes a ramped face oriented towards the shell.

12. The insertion device of claim 5, wherein the carriage further comprises at least one barb coupled to the platform and extending away from the platform towards the proximal opening.

13. The insertion device of claim 12, wherein the at least one barb comprises a plurality of barbs.

14. The insertion device of claim 12, wherein the at least one barb includes a front end spaced apart from the platform, the front end including an edge configured to interact with a boss positioned within the shell.

15. The insertion device of claim 6, wherein the housing comprising at least one guide slot on an inner wall of the shell, the at least one guide slot being configured to receive partially therein the at least one finger.

16. The insertion device of claim 4, wherein the trigger-type actuator further comprises:
a cap coupled to the housing;
a button positioned within the housing; and,
a trigger spring positioned between the button and a shoulder of the housing, the shoulder proximate the distal opening of the housing, wherein the trigger spring is configured to urge the button towards a distal end of the cap.

17. The insertion device of claim 16, wherein the carriage further comprises:
at least one trigger arm coupled to the platform and extending away from the platform towards the distal opening, and wherein the at least one trigger arm includes a trigger finger spaced distally from the platform, the at least one trigger finger including a ramped face oriented towards the shell;
and wherein the button further comprises:
a sleeve including a tapered face oriented away from the shell, the tapered face configured to interact with the ramped face of the at least one trigger finger so as to causes the at least one trigger finger to flex inward away from the shell when the button is depressed, thereby releasing the carriage.

18. The insertion device of claim 1, wherein the medical needle is part of a needle assembly.

19. A method of inserting a medical needle into a patient, the method comprising:
obtaining an insertion device of claim 1;
positioning the insertion device proximate a skin of a patient;
actuating the carriage to cause the carriage and the medical needle to move from the retracted position to the extended position.

20. A method of inserting a medical needle into a patient, the method comprising:
obtaining an insertion device of claim 17;
positioning the insertion device proximate a skin of a patient;
depressing the button to cause the carriage and the medical needle to move from the retracted position to the extended position.
